(19) United States
(12) Patent Application Publication
(10) Pub. No.: US 2025/0057571 A1
(43) Pub. Date: Feb. 20, 2025

(54) INTERBODY DEVICE WITH MEDIO-LATERAL EXPANSION CAPABILITY WITH ASSOCIATED INCREASE IN THE SURFACE AREA FOR USE IN POSTERIOR OR TRANSFORAMINAL INTERBODY LUMBAR FUSION

(71) Applicants: Tobias A. Mattei, Eureka, MO (US); Nicholas Musgrave, St. Louis, MO (US)

(72) Inventors: Tobias A. Mattei, Eureka, MO (US); Nicholas Musgrave, St. Louis, MO (US)

(21) Appl. No.: 18/785,208

(22) Filed: Jul. 26, 2024

Related U.S. Application Data
(60) Provisional application No. 63/519,732, filed on Aug. 15, 2023.

(51) Int. Cl. A61B 17/70

(52) U.S. Cl. CPC A61B 17/7071

(57) ABSTRACT

A device for interbody vertebral fusion is constructed of a cage supporting a plurality of upper plates on a top of the cage and supporting a plurality of lower plates on a bottom of the cage. An expansion mechanism is operatively connected between the cage and the upper plates and the lower plates. The expansion mechanism is operable to cause the plurality of upper plates to move to side-by-side relative positions increasing their combined surface area and to cause the plurality of lower plates to move to side-by-side relative positions increasing their combined surface area.

### CROSS-REFERENCE TO RELATED APPLICATIONS

This patent application claims priority from provisional patent application Ser. No. 63/519,732, which was filed on Aug. 15, 2023.

### BACKGROUND

Lumbar fusion is a surgical procedure that joins two adjacent vertebrae, or two adjacent bones in the lumbar spine, or the lower back. Lumbar fusion permanently stops movement between the two adjacent vertebrae of the lumbar spine, thereby reinforcing the back structure and adding stability and helping to prevent pain.

Posterior lumbar interbody fusion (PLIF) and transforaminal lumbar interbody fusion (TLIF) are two of the most common techniques for lumbar fusion.

The interbody fusion process involves removal of disc material from the disk space between two adjoining vertebrae, and the insertion of a device commonly referred to as a spinal cage or cage between the adjoining vertebrae in place of the removed disc material. The bone of the two adjoining vertebrae grows into the material of the cage and thereby the two adjoining vertebrae are fused together.

In posterior lumbar interbody fusion PLIF, the surgical approach is from behind the lumbar spine. The spinal disc between the two adjoining vertebrae is removed, and then replaced with the cage that is inserted in the space between the two adjoining vertebrae from the back of the spine. A biological response causes the bone of the two adjoining vertebrae to grow into the material of the cage and thereby join together the two adjoining vertebrae.

Transforaminal lumbar interbody fusion TLIF is a variation of PLIF where the surgeon approaches the disk space between the two adjoining vertebrae slightly to the side of and not directly from the back of the lumbar spine.

Although offering the benefit of interbody fusion and neural decompression through a single posterior approach, one of the disadvantages of PLIF and TLIF in comparison with other techniques (such as lateral lumbar interbody fusion LLIF or anterior lumbar interbody fusion ALIF) is that the size of the cage is limited due to the presence of the thecal sack and emerging nerve roots in the area between the two vertebrae to be joined. The limited size of the cage results in a reduction of the surface areas at the top and bottom of the cage that come into contact with the two vertebrae above and below the cage, respectively. The smaller surface areas of contact at the top and bottom of the interbody cage in TLIF and PLIF limits the ability of the cage to achieve adequate correction of segmental lordosis and sagittal imbalance, and is associated with an inherent larger risk of cage subsidence when compared with larger interbody cages which can be placed through the LLIF and ALIF techniques.

### SUMMARY

Cages with longitudinal expansion capability or expansion along the cranio-caudal length of the spine have been proposed in order to increase the ability of PLIF and TLIF cages to restore lumbar lordosis. However, no current cage has, as its main or substantial feature, a lateral expansion or medio-lateral expansion capability or expansion transverse to the spine with concomitant increase in the surface area of contact of the cage with the adjacent vertebral endplates, a feature which would increase the likelihood of achieving deformity correction and a successful fusion, while at the same time, reducing the rates of cage subsidence.

### SUMMARY OF THE INVENTION

The interbody cage device of this disclosure is designed for use in TLIF and PLIF procedures, either through open surgery or a minimally invasive approach. The interbody device comprises a cage with upper and lower mechanisms at the top and bottom of the cage, respectively, having overlapping plates or angularly adjacent plates. The plates of the mechanisms at the top and bottom of the cage are movable from their overlapped or angularly adjacent positions laterally in a medio-lateral expansion outward from the cage by sliding movement of the plates or pivoting movement of the plates relative to each other to achieve medio-lateral expansion of the plates at the top and bottom of the cage. The lateral expansion of the plates achieves an increase in the surface areas at the top and bottom of the cage opposing the adjoining vertebrae at the top and bottom of the cage. The cage can then be expanded in a longitudinal direction of the cage or a cranio-caudal direction of the spine to engage the adjoining vertebrae at the top and bottom of the cage.

Various different types of mechanisms are employed on the top and bottom of the PLIF or TLIF cage to achieve the medio-lateral expansion of the plates of the PLIF and TLIF interbody cages and the resultant increase in the surface areas at the top and bottom of the cages.

The features, functions and advantages of the interbody device that have been discussed above can be achieved independently in various embodiments or may be combined in yet other embodiments, further details of which can be seen with reference to the following description and drawings.

### DESCRIPTION OF THE DRAWINGS

FIG. 1 is a schematic representation of an end elevation view of an embodiment of the interbody device of this disclosure in which the upper and lower plates on the cage are movable laterally from overlapped relative positions to laterally adjacent side-by-side relative positions.

FIG. 2 is a schematic representation of an intermediate stage of lateral movement of the plates of the device of FIG. 1.

FIG. 3 is a schematic representation of an end elevation view of the device of FIG. 1 in which the upper and lower plates have been moved to their laterally adjacent side-by-side positions.

FIG. 4 is a schematic representation of an end elevation view of a further embodiment of the interbody device of this disclosure in which the upper and lower plates on the cage are movable in pivoting movements from general angled relative positions to laterally adjacent side-by-side relative positions.

FIG. 5 is a schematic representation of an intermediate stage of lateral pivoting movement of the plates of the device of FIG. 4.

FIG. 6 is a schematic representation of an end elevation view of the interbody device of FIG. 4 in which the upper and lower plates have been pivoted to their laterally adjacent side-by-side relative positions.

FIG. 7 is a schematic representation of an end elevation view of a still further embodiment of the interbody device of this disclosure in which the upper and lower plates on the cage are movable laterally from adjacent positions to laterally spaced side-by-side relative positions by extendable struts connected between the upper and lower plates.

FIG. 8 is a schematic representation of an end elevation view of the interbody device of FIG. 7 in which the upper and lower plates have been moved to their laterally spaced side-by-side relative positions.

### DETAILED DESCRIPTION

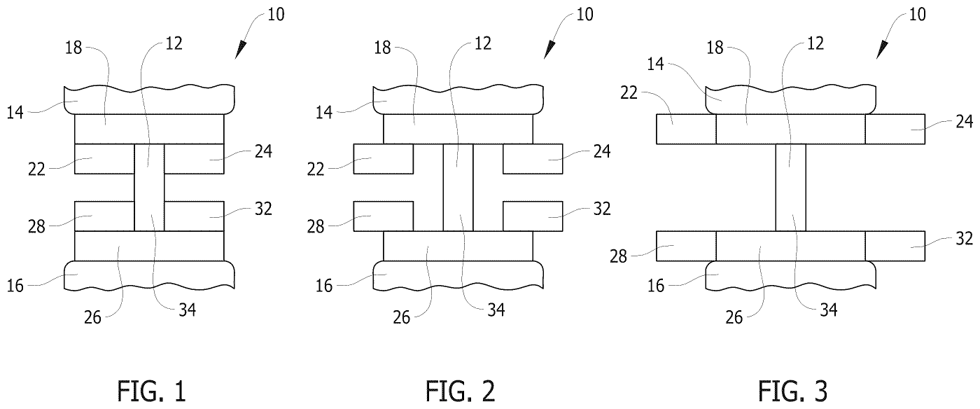

A first embodiment of the interbody device 10 of this disclosure is represented in FIG. 1, FIG. 2 and FIG. 3. The central component of the device 10 is the cage 12. The cage 12 is constructed in a manner and of materials that promote the growth of the adjacent vertebrae bone into the cage to achieve the fusion of the vertebrae. The cage 12 has a longitudinal dimension between a top of the cage and a bottom of the cage. The cage longitudinal dimension is substantially the same cranio-caudal dimension as the disc material removed from between the upper or top vertebrae 14 and the lower or bottom vertebrae 16. The cage 12 also has a lateral dimension between a first side of the cage and a second side of the cage (the left and right sides of the cage as viewed in FIG. 1) that is perpendicular to the longitudinal dimension. The cage lateral dimension is substantially the same as the medio-lateral dimension of the disc material removed from between the upper or top vertebrae 14 and the lower or bottom vertebrae 16. The cage 12 also has a width dimension or a depth dimension between a front of the cage and a rear of the cage. The depth dimension extends into the cage 12 as viewed in FIG. 1 and FIG. 2 and is substantially the same dimension as the disc material removed from between the upper or top vertebrae 14 and the lower or bottom vertebrae 16. In this manner, the cage 12 is dimensioned to occupy substantially the same volume between the upper vertebrae 14 and the bottom vertebrae 16 that was occupied by the disc material removed from between the vertebrae.

A plurality of upper plates 18, 22, 24 are connected to the top of the cage 12 and a plurality of lower plates 26, 28, 32 are connected to the bottom of the cage 12. The plates are constructed in a manner and of a material that promotes bone growth into the plates. The plates 18, 22, 24 and 26, 28, 32 are connected to the top and bottom of the cage 12, respectively, by mechanisms incorporated into the cage 12 that enable sliding movement of adjacent plates on the cage 12.

Prior to insertion between adjacent vertebrae 14, 16, the inter body device 10 is adjusted to its compact configuration represented in FIG. 1. In the compact configuration the upper plates 18, 22, 24 and the lower plates 26, 28, 32 are adjusted to their overlapping and stacked relative positions. On the cage 12 represented in FIGS. 1, 2 and 3, each of the plates 18, 22, 24 and 26, 28, 32 has a rectangular configuration of substantially the same longitudinal, lateral and width dimensions. Adjacent plates are connected by tongue and groove connections that extend laterally and enable relative sliding movement of adjacent plates laterally from the overlapping and stacked relative positions on the top and bottom of the cage 12 represented in FIG. 1 to the side-by-side relative positions represented in FIG. 3. Other equivalent connections that enable the sliding movement of the plates 18, 22, 24 and 26, 28, 32 on the top and bottom of the cage 12 could be employed.

A rack and pinion mechanism incorporated in the cage 12 could be operatively connected between adjacent plates to cause the sliding and separating movements of the plates in response to rotation of a pinion gear. Alternatively, a compressed spring device could be connected between adjacent plates with a spring of the device being compressed when the adjacent plates are in their overlapping and stacked relative positions represented in FIG. 1. By operation of an actuator of the compressed spring device, the compressed spring can be released to move adjacent plates in the sliding, separating relative movements represented in FIG. 2 to the side-by-side configuration of the adjacent plates represented in FIG. 3. Still further, other equivalent types of drive mechanisms connected between the stacked adjacent plates 18, 22, 24 and 26, 28, 32 could be used to control movement of the plates laterally from their relative stacked positions represented in FIG. 1 to their side-by-side separated positions represented in FIG. 3.

As stated earlier, prior to insertion between the adjacent vertebrae 14, 16, the interbody device 10 is adjusted to its compact configuration. The device of FIG. 1 is intended to be inserted through a standard posterior surgical approach similar to that employed in the insertion of other PLIF and TLIF devices. The standard open surgical technique for these types of procedures involves a posterior midline incision, subperiostal paraspinal muscle dissection, laminectomy (either full laminectomy with bilateral facetectomies or limited foraminotomy), microdiscectomy, vertebral endplate preparation and interbody device insertion. The standard technique for a minimally invasive (MIS) lumbar fusion approach involves percutaneous placement of pedicle screws (either guided by fluoroscopy, navigation or robotics), a paramedian (Wiltse) approach with dissection between the multifidus and erector spinalis muscle on one side, placement of tubular retractors or other type of MIS retractor, limited foraminotomy, unilateral microdiscectomy or endoscopic tubular discectomy, endplate preparation and interbody device placement.

FIG. 1 is a representation of the orientation of the interbody device 10 once inserted into the disk space (a process similar to the currently used technique) between an upper spinal vertebrae 14 and a lower, adjacent spinal vertebrae 16. During the initial insertion of the device 10, the upper plates 18, 22, 24 and lower plates 26, 28, 32 of the device 10 are in their overlapped or stacked relative positions on the top and bottom of the cage 12, respectively. Once inserted, an instrument manipulated by the surgeon would exert a torque upon an expansion mechanism 34 incorporated in the cage 12 of the interbody device 10 which is operable to deploy the plates. For example, a clamp or grasper that engages a rotary actuator of the interbody device 10 and manually rotates the actuator to cause the plates 18, 22, 24 and 26, 28, 32 to move laterally apart on the top and bottom of the cage 12 and in the medio-lateral direction relative to the upper 14 and lower 16 adjacent spinal vertebrae. Alternatively, a clamp or grasper could be used to manually operate an actuator of a spring device such as that described earlier. A simple surgical device in the form of a screwdriver could also be used to manually rotate or operate an actuator of the interbody device 10 to initiate the sliding, separating movement of the plates 18, 22, 24 and 26, 28, 32 on the top and bottom of the cage 12. Any equivalent type of actuator could be used to operate the sliding, separating movement of the plates 18, 22, 24 and 26, 28, 32 on the cage 12.

To control the relative positions of the plates 18, 22, 24 and 26, 28, 32 during their lateral sliding, separating movements to position the plates in their side-by-side relative positions, the adjacent plates could have mechanical features such as tongue and groove connections or other equivalent connections that control the sliding and separating movement of the plates along linear paths.

As represented in FIG. 2, operation of the expansion mechanism 34 causes the upper plates 18, 22, 24 and the lower plates 26, 28, 32 to move laterally relative to each other and the cage 12 to their expanded relative positions. In the expanded positions the upper plates 18, 22, 24 cover additional surface area of the endplate of the upper vertebrae 14 at the top of the cage 12, and the lower plates 26, 28, 32 cover additional surface area of the endplate of the lower vertebrae 16 at the bottom of the cage 12.

As represented in FIG. 1, initially the upper plates 18, 22, 24, and lower plates 26, 28, 32 are positioned within the lateral and depth dimensions of the cage 12 allowing for the smallest possible dimensions for interbody device 10 insertion and, therefore, minimizing the need for retraction of neural structures and possible nerve injury. To achieve this packaging, the plates 18, 22, 24 and 26, 28, 32 of the interbody device 10 are stacked on top of each other within the lateral and depth dimensions of the cage 12 as represented in FIG. 1. At the time of expansion, the plates 18, 22, 24 and 26, 28, 32 would slide in the lateral direction crossing beyond the initial lateral dimension of the cage 12 to positions outside the lateral dimension of the cage to positions overlapping each other, like roofing shingles. In the end result represented in FIG. 3, the expanded plates 18, 22, 24 and 26, 28, 32 are positioned side-by-side and will cover more surface areas of the upper 14 and lower 16 vertebral end plates, ultimately promoting greater bone fusion rates. The interbody device 10 is then operated in substantially the same manner as known cages with cranio-caudal expansion capability to expand in the longitudinal direction of the device or in the cranio-caudal direction of the spine.

This technique can be performed either unilaterally or bilaterally. The greater surface area provided as represented in FIG. 3 by the interbody device 10 with medio-lateral expansion relative to the upper 14 and lower 16 vertebrae will also increase the capacity of the device for correction of sagittal and coronal deformity and will reduce the chances of cage subsidence, a feared complication of such type of procedures.

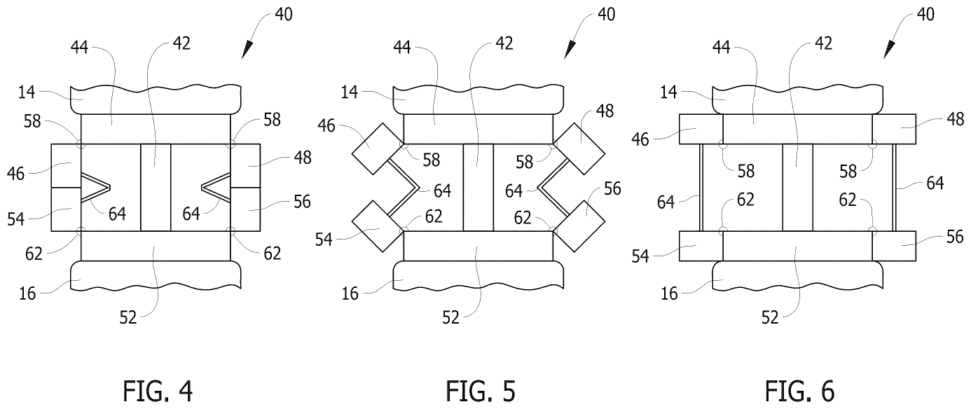

FIG. 4 is a representation of a further embodiment of the interbody device 40 constructed in a similar manner to and of the same materials as the interbody device 10 of FIGS. 1, 2 and 3. In the interbody device 40 represented in FIGS. 4, 5 and 6 the central component of the device 40 is the cage 42. The cage 42 is constructed in a manner and of materials that promote the growth of the adjacent vertebrae bone into the cage to achieve the fusion of the vertebrae. The cage 42 has a longitudinal dimension between a top of the cage and a bottom of the cage. The cage longitudinal dimension is substantially the same cranio-caudal dimension as the disc material removed from between the upper or top vertebrae 14 and the lower or bottom vertebrae 16. The cage 42 also has a lateral dimension between a first side of the cage and a second side of the cage that is perpendicular to the longitudinal dimension. The cage lateral dimension is substantially the same as the medio-lateral dimension of the disc material removed from between the upper vertebrae 14 and the lower vertebrae 16. The cage 42 also has a width dimension or a depth dimension between a front of the cage and a rear of the cage. The depth dimension extends into the cage 42 as viewed in FIGS. 4, 5 and 6 and is substantially the same dimension as the disc material removed from between the upper vertebrae 14 and the lower vertebrae 16. In this manner, the cage 42 is dimensioned to occupy substantially the same volume between the upper vertebrae 14 and the bottom vertebrae 16 that was occupied by the disc material removed from between the vertebrae.

A plurality of upper plates 44, 46, 48 are connected to the top of the cage 42 and a plurality of lower plates 52, 54, 56 are connected to the bottom of the cage 42. The plates are constructed in a manner and of a material that promotes bone growth into the plates. The plates 44, 46, 48 and 52, 54, 56 are connected to the top and bottom of the cage 42, respectively by mechanisms that enable laterally outward pivoting movement of adjacent plates on the cage 42.

Pivot connections 58, 62 enable achieving medio-lateral expansion of the upper plates 44, 46, 48 and the lower plates 52, 54, 56 of the interbody device 42. The upper plates 44, 46, 48 are connected by pivot connections 58 and the lower plates 52, 54, 56 are connected by pivot connections 62. The interbody device 40 of FIGS. 4, 5 and 6 employs an umbrella type actuation expansion mechanism 64 incorporated into the cage 42 to control movement of the plates.

As the interbody device 40 is inserted between adjoining upper 14 and lower 16 vertebrae, the upper plates 44, 46, 48 and lower plates 52, 54, 56 are connected through supporting expandable rods attached to a main cranio-caudal expanding strut of the expansion mechanism 64. The supporting expandable rods and the main strut are assembled together in a manner similar to that of an umbrella to form the expansion mechanism 64 of the interbody device 40. As the strut of the device 40 is expanded in the cranio-caudal aspect or along the length of the spine, the rods 64 move the upper plates 44, 46, 48 to pivot laterally outward from the cage 42 and relative to each other so that they are moved and pulled outward to cover the medio-lateral surface of the upper vertebrae 14 as represented in FIG. 5. Simultaneously, the rods move the lower plates 52, 54, 56 to pivot laterally outward relative to each other so that they are moved and pulled outward to cover the medio-lateral surface of the lower vertebrae 16 as represented in FIG. 5. Alternatively, the expansion mechanism 64 could be operable by a rack and pinion actuator, a compressed spring actuator or any other equivalent type of actuator as those described earlier. The mechanism 64 does not necessarily need to rely on cranio-caudal expansion as the blades could be initially contained by a locking mechanism which could be released through application of a torque by an insertion handle. In such a rendition of this embodiment, the plates 44, 46, 48 and 52, 54, 56 could be deployed laterally first and then locked in place. The interbody device 40 is then operated in substantially the same manner as known cages with cranio-caudal expansion capability to expand in the longitudinal direction of the device or in the cranio-caudal direction of the spine.

Such an embodiment, when combined with currently available cranio-caudal technology for cage expansion, would allow greater forces to be applied to the upper and lower vertebrae and plates during cranio-caudal expansion or expansion along the length of the spine.

This embodiment of the interbody device 40 could be packaged with the upper plates 44, 46, 48 and lower plates 52, 54, 56 folded along the outside of the device 42, with the laterally inferior plates folded outside of the superior plates or with the laterally superior plates folded outside the inferior plates.

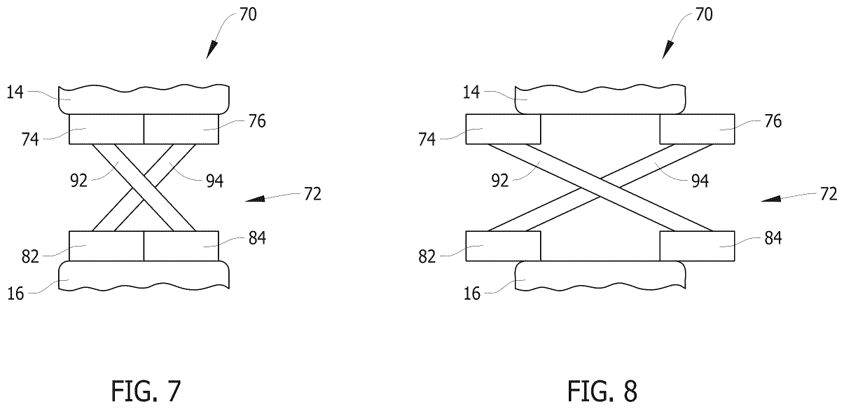

FIG. 7 is a representation of a further embodiment of the interbody device 70 through which medio-lateral expansion of the upper plates 74, 76 and lower plates 82, 84 can be achieved by involving a combination of the umbrella and overlapping plate mechanism, with an X shaped expandable strut structure expansion mechanism 88. As represented in FIG. 7, the interbody device 70 this comprised of a cage 72 with two upper plates 74, 76 connected to the top of the cage 72 and two lower plates 82, 84 connected to the bottom of the cage 72. The left side upper plate 74 is connected by an expandable strut 92 incorporated in the cage 72 to the right side lower plate 84. The right side upper plate 76 is connected by an expandable strut 94 incorporated in the cage 72 to the left side lower plate 82. Expansion of the struts 92, 94 can be achieved through the insertion of a medical instrument that can be manually manipulated to operate the expansion mechanism of the struts 92, 94. The operation of the expansion mechanism expands the lengths of the struts 92, 94 and results in the lateral movement of the upper plates 74, 76 away from each other and away from the cage 72 and the lateral movement of the lower plates 82, 84 away from each other and away from the cage 72. This results in the expansion of the interbody device 70 in the medio-lateral plane until the upper surface of the device 70 defined by the upper plates 74, 76 and the lower surface of the device 70 defined by the lower plates 82, 84 are located in the same cranio-caudal planes of the upper vertebrae 14 and the lower vertebrae 16 as represented in FIG. 8.

Each of the cage devices 12, 42, 72 described above is equipped with standard technology to insert bone graft material into the cage devices to thereby increase the rate of fusion of the vertebrae into the devices.

Equivalent devices of those described deploy some form of plates medially and/or laterally, while being initially located within the initial width of the interbody device. The plates can be deployed in isolation to achieve only medio-lateral expansion or in combination with current technology for cranio-caudal expansion. In the case of combined expansion, the medio-lateral stage of expansion may occur before or after cranio-caudal expansion. The devices may employ any form of ratcheting technique or torquing mechanism or any mode of expansion, performed either manually, through the insertion handle or through other mechanisms. The novelty of the devices is in the medio-lateral expansion of a standard TLIF/PLIF interbody device in order to increase the top and bottom surface areas covered by the interbody device and, therefore, increase its capacity of deformity correction and reducing the rates of cage subsidence while also increasing the likelihood of bone fusion.

As various modifications could be made in the construction of the interbody device and its method of operation herein described and illustrated without departing from the scope of the invention, it is intended that all matter contained in the foregoing description or shown in the accompanying drawings shall be interpreted as illustrative only rather than limiting. Thus, the breadth and scope of the present disclosure should not be limited by any of the above-described exemplary embodiments but should be defined only in accordance with the following claims appended hereto and their equivalents.

1. A device for interbody vertebrae fusion comprising:
a cage;
a plurality of upper plates operatively connected to a top of the cage;
a plurality of lower plates operatively connected to a bottom of the cage; and
an expansion mechanism operatively connected to the plurality of upper plates and the plurality of lower plates, the expansion mechanism being operable to cause the plurality of upper plates to move relative to each other to side-by-side positions and to cause the plurality of lower plates to move relative to each other to side-by-side positions.

2. The device of claim 1, further comprising:
the expansion mechanism being operable to cause the plurality of upper plates to move from overlapped, stacked relative positions of the upper plates to the side-by-side relative positions of the upper plates; and
the expansion mechanism being operable to cause the plurality of lower plates to move from overlapped, stacked relative positions of the lower plates to the side-by-side relative positions of the lower plates.

3. The device of claim 1, further comprising:
the expansion mechanism being operable to cause the plurality of upper plates to pivot from angular orientations between adjacent upper plates to the side-by-side relative positions of the upper plates; and
the expansion mechanism being operable to cause the plurality of lower plates to pivot from angular orientations between adjacent lower plates to the side-by-side relative positions of the lower plates.

4. The device of claim 1, further comprising:
the plurality of upper plates and the plurality of lower plates being constructed of a material that promotes bone growth into the material of the plates.

5. The device of claim 1, further comprising:
the plurality of upper plates is operatively connected for relative sliding movement between adjacent plates of the plurality of upper plates; and
the plurality of lower plates is operatively connected for relative sliding movement between adjacent plates of the plurality of lower plates.

6. The device of claim 1, further comprising:
the plurality of upper plates is operatively connected for relative pivoting movement between adjacent plates of the plurality of upper plates; and
the plurality of lower plates is operatively connected for relative pivoting movement between adjacent plates of the plurality of lower plates.

7. The device of claim 1, further comprising:
the plurality of upper plates is constructed of a material that promotes bone growth into the material of the upper plates; and
the plurality of lower plates is constructed of a material that promotes bone growth into the material of the plurality of lower plates.

8. A device for interbody vertebrae fusion between an upper vertebra and a lower vertebra comprising;
a cage, the cage having a longitudinal dimension that is configured for insertion between the upper vertebrae and the lower vertebrae, the cage having a top of the cage and a bottom of the cage that are longitudinally spaced;
the cage having a lateral dimension, the lateral dimension being mutually perpendicular with the longitudinal dimension;
a plurality of upper plates on the top of the cage, the plurality of upper plates on the top of the cage being movable on the top of the cage; and
a plurality of lower plates on the bottom of the cage, the plurality of lower plates on the bottom of the cage being movable on the bottom of the cage.

9. The device of claim 8, further comprising:
the plurality of upper plates on the top of the cage being movable in laterally sliding movements on the top of the cage.

10. The device of claim 9, further comprising:
the plurality of lower plates on the bottom of the cage being movable in laterally sliding movements on the bottom of the cage.

11. The device of claim 10, further comprising:
tongue and groove connections connecting adjacent plates of the plurality of upper plates and adjacent plates of the plurality of lower plates.

12. The device of claim 8, further comprising:
the plurality of upper plates on the top of the cage being movable in pivoting movements on the top of the cage.

13. The device of claim 12, further comprising:
the plurality of lower plates on the bottom of the cage being movable in pivoting movements on the bottom of the cage.

14. The device of claim 13, further comprising:
pivot connections connecting adjacent plates of the plurality of upper plates and the plurality of lower plates.

15. The device of claim 8, further comprising:
the plurality of upper plates is constructed of a material that promotes bone growth into the material of the upper plates; and
the plurality of lower plates is constructed of a material that promotes bone growth into the material of the plurality of lower plates.

16. A method of fusing an upper vertebrae and an adjacent lower vertebrae comprising:
surgically positioning an interbody device between the upper vertebrae and the lower vertebrae.
moving a plurality of upper plates on the interbody device laterally to positions adjacent the upper vertebrae; and
moving a plurality of lower plates on the interbody device laterally to positions adjacent the lower vertebrae.

17. The method of claim 16, further comprising:
moving the plurality of upper plates from overlapped, stacked relative positions of the upper plates to side-by-side relative positions of the upper plates; and
moving the plurality of the lower plates from overlapped, stacked relative positions of the lower plates to side-by-side relative positions of the lower plates.

18. The method of claim 16, further comprising:
pivoting the plurality of upper plates from angular orientations between adjacent upper plates to side-by-side relative positions of the upper plates; and
pivoting the plurality of lower plates from angular orientations between adjacent lower plates to side-by-side relative positions of the lower plates.

19. The method of claim 16, further comprising:
removing disk material from between the upper vertebrae and the lower vertebrae prior to surgically positioning the interbody device between the upper vertebrae and the lower vertebrae.

20. The method of claim 16, further comprising:
promoting bone growth into the plurality of upper plates and into the plurality of lower plates.
